(19) United States
(12) Patent Application Publication
(10) Pub. No.: US 2007/0134214 A1
(43) Pub. Date: Jun. 14, 2007

(54) Inactivated host cell delivery of polynucleotides encoding immunogens

(76) Inventor: Feng Xu, Walnut Creek, CA (US)

Correspondence Address: NOVARTIS VACCINES AND DIAGNOSTICS INC., CORPORATE INTELLECTUAL PROPERTY R338, P.O. BOX 8097, Emeryville, CA, 94662-8097, US

(21) Appl. No.: 10/567,940

(22) PCT Filed: Aug. 12, 2004
(86) PCT No.: PCT/US04/25914

§ 371 Date: Sep. 27, 2006

Related U.S. Application Data
(60) Provisional application No. 60/494,818, filed on Aug. 13, 2003.

(51) Int. Cl. A61K 48/00; A61K 39/02

(52) U.S. Cl. 424/93.21; 424/200.1

(57) ABSTRACT

Provided herein methods for in vivo delivery of an immunogen. In particular, the methods comprise administering an inactivated host cell to a mammal, wherein the host cell comprises a polynucleotide encoding an immunogen. After administration, the immunogen is then expressed in vivo by the mammal.

### FIELD OF THE INVENTION

The invention relates to inactivated host cell comprising polynucleotide encoding one or more immunogens and methods of administering these inactivated host cells to mammals for in vivo expression of the immunogens.

### BACKGROUND

Various gene delivery techniques have been developed over the years to deliver polynucleotides to mammalian cells for in vivo expression. Such techniques have been used for delivery of potential vaccine antigens as well as various therapeutic proteins.

For example, live bacteria have been used to deliver plasmids encoding potential immunogens to mammals for in vivo expression of an encoded protein. Alternatively, polynucleotides encoding potential immunogens have been incorporated into open reading frames within bacterial genomes. Vectors and promoters tailored to specific bacterial host cells have been used to facilitate and increase the expression of a protein of interest. See, for example, U.S. Pat. No. 6,500,419 (describing delivery of a bacterial RNA vector from a live bacteria to eukaryotic cells).

The use of such live bacterial systems as DNA vaccine delivery systems raises some safety issues. Efforts have been made to identify suitably non-pathogenic or attenuated bacteria for these delivery systems. See, e.g., Xu et al., “Immunogenicity of an HIV-1 gag DNA vaccine carried by attenuated Shigella” Vaccine (2003) 21:644-648. However, a need remains for safer and more efficient mechanisms for gene delivery, including for vaccines.

Applicants have unexpectedly and surprisingly found that an immune response can be elicited via the administration of an inactivated host cell comprising a polynucleotide encoding an immunogen. Despite the inactivation of the host cell prior to administration to the mammal, the encoded immunogen is expressed in vivo, apparently by the mammalian cellular machinery. The resulting immune response is specific to the encoded immunogen.

The invention further provides a more efficient and economical method for the delivery of DNA vaccines which does not require the isolation of a specific DNA plasmid from a recombinant host cell.

### SUMMARY OF THE INVENTION

The invention provides a method for in vivo expression of an immunogen comprising administering an inactivated host cell to a mammal, wherein said host cell comprises a polynucleotide encoding an immunogen. After administration, the immunogen is then expressed in vivo by the mammalian cells.

The host cell is inactivated and unable to replicate or to use its own cell machinery to express the encoded immunogen. Preferably, the host cell is inactivated by heat treatment, UV light exposure, or hydrogen peroxide treatment.

The inactivated host cell may comprise a plasmid comprising the polynucleotide encoding the immunogen. Alternatively, the immunogen may be incorporated into the host cell genome. Suitable promoters and vectors may be chosen to direct the expression of the immunogen.

The encoded immunogen preferably generates an immune response in the mammal upon in vivo expression. Accordingly, the invention includes a method of generating an immune response in a mammal comprising administering an inactivated host cell to said mammal, said inactivated host cell comprising a polynucleotide encoding an immunogen.

The invention also provides a composition comprising an inactivated host cell of the invention. The composition may further comprise a pharmaceutically acceptable solution and/or a pharmaceutically acceptable carrier.

The invention also provides a method of making an inactivated host cell comprising (i) transforming a host cell with a vector comprising a polynucleotide encoding an immunogen and (ii) inactivating the host cell. Methods of inactivating the host cell include heat treatment, UV light exposure, hydrogen peroxide, or other standard methods known in the art.

Preferably, the inactivated host cells are detoxified. For example, a bacterial host cell may be genetically manipulated to carry a different set of lipopolysaccharides (LPS) synthesis genes, for making a nonreactogenic LPS molecule with a desirable composition. Many studies have shown that the composition of the lipid portion in the LPS determines the level of toxicity in an animal, and that LPS molecules from some bacterial cell walls are more reactogenic than others. See, e.g., ref. 1.

### DETAILED DESCRIPTION OF THE INVENTION

The invention relates to inactivated host cells containing polynucleotides encoding an immunogen and methods of administering the host cell to a mammal for in vivo expression of the immunogen. In order to facilitate an understanding of the invention, selected terms used in the application will be discussed below.

The terms “polypeptide”, “protein” and “amino acid sequence” as used herein generally refer to a polymer of amino acid residues and are not limited to a minimum length of the product. Thus, peptides, oligopeptides, dimers, mulimers, and the like, are included within the definition. Both full-length proteins and fragments thereof are encompassed by the definition. Minimum fragments of polypeptides useful in the invention can be at least 3, 4, 5, 6, 7, 8, 9, 10, 11, 12, 13, 14, or even 15 consecutive amino acids. Typically, polypeptides useful in this invention can have a maximum length suitable for the intended application. Preferably, the polypeptides of the invention are not longer than 300 consecutive amino acids (i.e., no longer than 300, 290, 280, 270, 260, 250, 240, 230, 220, 210, 200, 190, 180, 170, 160, 150, 140, 130, 120, 110, 100, 90, 80, 70, 60, 50, 40, or 30). Generally, the maximum length is not critical and can easily be selected by one skilled in the art.

Reference to polypeptides and the like also includes derivatives of the amino acid sequences of the invention. Such derivatives can include postexpression modifications of the polypeptide, for example, glycosylation, acetylation, phosphorylation, and the like. Amino acid derivatives can also include modifications to the native sequence, such as deletions, additions and substitutions (generally conservative in nature), so long as the protein maintains the desired activity. These modifications may be deliberate, as through site-directed mutagenesis, or may be accidental, such as through mutations of hosts which produce the proteins or errors due to PCR amplification. Furthemlore, modifications may be made that have one or more of the following effects: reducing toxicity; facilitating cell processing (e.g., secretion, antigen presentation, etc.); and facilitating presentation to B-cells and/or T-cells.

“Fragment” or “Portion” as used herein refers to a polypeptide consisting of only a part of the intact full-length polypeptide sequence and structure as found in nature. For instance, a fragment can include a C-terminal deletion and/or an N-terminal deletion of a protein.

The term “polynucleotide”, as known in the art, generally refers to a nucleic acid molecule. A “polynucleotide” can include both double- and single-stranded sequences and refers to, but is not limited to, cDNA from viral, prokaryotic or eukaryotic mRNA, genomic RNA and DNA sequences from viral (e.g. RNA and DNA viruses and retroviruses) or prokaryotic DNA, and especially synthetic DNA sequences. The term also captures sequences that include any of the known base analogs of DNA and RNA, and includes modifications such as deletions, additions and substitutions (generally conservative in nature), to the native sequence, so long as the nucleic acid molecule encodes a therapeutic or antigenic protein. These modifications may be deliberate, as through site-directed mutagenesis, or may be accidental, such as through mutations of hosts that produce the antigens. Modifications of polynucleotides may have any number of effects including, for example, facilitating expression of the polypeptide product in a host cell.

A polynucleotide can encode an immunogenic protein or polypeptide (“immunogen”). Depending on the nature of the polypeptide encoded by the polynucleotide, a polynucleotide can include as little as 10 consecutive nucleotides, e.g., where the polynucleotide encodes an antigen. Preferably, the polynucleotide comprises at least 20 consecutive nucleotides (e.g., at least 20, 25, 30, 45, 50, 55, 60, 65, 70, 75, 80, 85, 90, 95, 100, 150, 200, 250, or 300).

Inactivated Host Cell

The invention relates to inactivated host cells comprising polynucleotides encoding an immunogen.

Host cells suitable for use in the invention include bacterium, yeast, mycobacterium, viruses, insects or other any organism capable of including the polynucleotide encoding the immunogen either integrated within its genome or within a plasmid, or a DNA or RNA replicon. Preferably, the host cell is a bacterial host cell.

Bacterial host cells suitable for use in the invention include E. coli, Shigella spp., Bordella spp., Salmonella spp., Bacillus spp., Streptococcus spp., Mycobacteria spp. or other bacterial species, or other microorganisms which can harbor plasmids or DNA or RNA replicons. Preferably, the bacteria host cell of the invention is an E. coli or a Shigella or an attenuated bacterial strain already used in humans, including Mycobacterium bovis (BCG) and Salmonella typhi TY21a.

Fungi and yeast host cells suitable for use in the invention include Absidia spp., Acremonium spp., Actinomadura spp., Alternaria spp., Apophysomyces spp., Arthrinium spp., Arthrographis spp., Aspergillus spp., Aureobasidium spp., Basidiobolus spp., Beauveria spp., Bipolaris spp., Blastomyces spp., Blastoschizomyces spp., Botrytis spp., Candida spp., Chaetomium spp., Chrysosporium spp., Cladophialophora spp., Cladosporium spp., Coccidioides spp., Conidiobolus spp., Cryptococcus spp., Cunninghamella spp., Curvularia spp., Dermatophytes, Emmonsia spp., Epicoccum spp., Epidermophyton spp., Exophiala spp., Fonsecaea spp., Fusarium spp., Geotrichum spp., Gliocladium spp., Graphium spp., Hansenual spp., Helminthosporium spp., Histoplasma spp., Hortaea werneckii, Kluyveromyces spp., Lacazia spp., Leptosphaeria spp., Madurella spp., Malassezia spp., Malbranchea spp., Microsporum spp., Mucor spp., Neotestudina spp., Nigrospora spp., Nocardia spp., Nocardiopsis spp., Paecilomyces spp., Paracoccidioides spp., Penicillium spp., Phaeococcomyces spp., Phialophora spp., Phoma spp., Piedraia spp., Pichia spp., Pneumocystis spp., Pseudcallescheria spp., Pyrenochaeta spp., Rhizomucor spp., Rhizopus spp., Rhodotorula spp., Saccharomyces spp., Schizosaccharomyces spp., Scedosporium spp., Scopulariopsis spp., Sepedonium spp., Sporobolomyces spp., Sporothrix spp., Sporotrichum spp., Stachybotrys spp., Stemphylium spp., Streptomyces spp., Syncephalastrum spp., Trichoderma spp., Trichophyton spp., Trichosporon spp., Trichothecium spp., Ulocladium spp., Ustilago spp., Verticillium spp., Wangiella spp., Yarrowia spp., and Zygomnycetes. Preferably, the fungi or yeast host cells of the invention are selected from the group consisting of Saccharomyces spp. and Streptomyces spp.

Inset host cells suitable for use in the invention, for example with baculovirus expression vectors include, Aedes aegypti, Autographa californica, Bombyx mori, Drosophila melanogaster, Spodopterafrugiperda, and Trichoplusia ni.

The invention includes a method of making an inactivated host cell comprising (i) transforming a host cell with a polynucleotide encoding an immunogen and (ii) inactivating the host cell.

In one embodiment, the host cells of the invention comprise a plasmid construct comprising the encoded immunogen. Recombinant vectors and methods of inserting a gene into a plasmid construct for recombinant expression are known in the art. For example, the polynucleotide sequence encoding the immunogen may be cloned into Sall and EcoRI restriction sites of the eukaryotic expression vector pCMVKm2. This vector contains a cytomegalovirus (CMV) immediate-early enhancer/promoter and a bGH terminator. The resulting plasmid can be transformed into a host cell, and the host cell inactivated.

The host cells of the invention are inactivated, meaning they are no longer capable of replication. Preferably, they are no longer capable of using their host cell machinery to express the encoded immunogen. Suitable means of inactivating the host cells of the invention including heat, UV light exposure, hydrogen peroxide, or other standard methods known in the art.

The host cells of the invention may be modified to make them more suitable for pharmaceutical administration. For example, the host cell may be detoxified. Suitable means of detoxification include chemical or enzymatic treatments, genetic manipulation to remove or decrease expression of a toxin protein (peptide), lipid, sugar or combinations thereof, and to change the compositions of these molecules etc. For example, a bacterial host cell may be genetically manipulated to remove or decrease expression of any LPS synthesis genes. Removal and modification of bacterial lipopolysaccharide (LPS) genes (such as, for example, the msbB gene) to detoxify bacteria have been disclosed in the art. As an another example, where the host cell is a gram-positive bacteria, the bacterial genes encoding for peptidoglycan synthesis may be removed or modified to provide for a less reactogenic or less toxic host cell. Accordingly, the invention includes an inactivated host cell which has been detoxified or which has decreased toxicity. Preferably,the host cell is a bacterial host cell with a modified LPS gene (such as the msbB gene). Preferably, this host cell has reduced or modified LPS synthesis.

Immunogen

Immunogens suitable for use in the invention include any polypeptide capable of eliciting an immune response. Immunogens of the invention may include a molecule containing one or more epitopes (either linear, conformational or both) that will stimulate a host's immune system to make a humoral and/or cellular antigen-specific response. Normally, an epitope will include between about 3-15, generally about 5-15 amino acids. A B-cell epitope is normally about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids.

As used herein, an “immune response” to an immunogen or antigen of the invention includes the development in a subject of a humoral and/or a cellular immune response to an immunogen or antigen present in the composition of interest. For purposes of the present invention, a “humoral immune response” refers to an immune response mediated by antibody molecules, including secretory (IgA) or IgG molecules, while a “cellular immune response” is one mediated by T-lymphocytes and/or other white blood cells.

One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. In addition, a chemokine response may be induced by various white blood or endothelial cells in response to an administered antigen.

Thus, an “immune response” as used herein may be one that stimulates CTLs, and/or the production or activation of helper T-cells. The production of chemokines and/or cytokines may also be stimulated. The antigen of interest may also elicit an antibody-mediated immune response. Hence, an immunological response may include one or more of the following effects: the production of antibodies (e.g., IgA or IgG) by B-cells ; and/or the activation of suppressor, cytotoxic, or helper T-cells and/or (*T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

Immunogens suitable for use in the invention include immunogens derived from viruses, bacteria, parasites, and cancer antigens.

Examples of bacterial antigens suitable for use in the invention include bacterial antigens derived from Neiserria meningitidis (including serogroup A, B, C, W135 and/or Y), Streptococcus pneumoniae, Streptococcus agalactiae (Group B Streptococcus), Streptococcus pyogenes (Group A streptococcus), Enterococcusfaecalis, Helicobacter pylori (including Cag, Vac, Nap and Urease), Bordetella pertussis, Staphylococcus aureus, Haemophilus influenzae, non-typeable H. influenzae, Staphylococcus epidermis, Staphylococcus saprophyticus, Pseudomonas aeruginosa, Bacillus anthracis, Moraxella catarrhalis, Neiserria gonorrhoeae, Chlamydia pneumoniae, Chlamydia trachomatis, Porphyromonas gingivalis, Clostridium tetani (tetanus toxoid), Corynebacterium diphtheriae (diphtheria toxoid), Borrelia burgdorferi, Treponema pallidum, Yersinia pestis, Yersinia enterocolitica, Yersinia pseudotuberculosis, Mycobacterium tuberculosis, Legionella pneumophila, Escherichia. coli, Vibrio cholerae, Salmonella typhi, Salmonella typhimurium, and Listeria monocytogenes.

Examples of viral antigens suitable for use in the invention include influenza (including hemagglutinin and or neuraminidase surface proteins), Respiratory Syicytial virus (RSV), Parainfluenza (PIV), polio antigens such as OPV or, preferably IPV, measles, mumps, rubella, rabies, yellow fever virus, Japanese encephalitis virus, Dengue virus, tick borne encephalitis virus, West Nile virus, HIV (including HIV-1 or HIV-2) (such as gag, env, pol, tat, nef, rev, vpu, and miniproteins), rotavirus (such as VP4, VP6, or VP7), parvovirus, (such as parvovirus B19), coronavirus, including a SARS virus (i.e., spike protein), hepatitis A virus (HAV), hepatitis B virus (HBV) (such as the surface and/or core antigens), hepatitis C virus (HCV) (such as E1, E2, E1/E2, NS345 polyprotein, NS 345-core polyprotein, core, and/or peptides from the nonstructural regions), heptatitis E virus, herpes virus, such as gD or derivaties thereof or Immediate Early protein such as ICP27 from HSV1 or HSV2, cytomegalovirus (such as gB or derivatives thereof), Epstein Barr Virus (such as gp350 or derivatives thereof).

Examples of parasitic antigens suitable for use in the invention include antigens derived from parasites that cause malaria. These include antigens derived from Plasmodia falciparum.

Examples of cancer antigens suitable for use in the invention include antigens recognized by CD8+ lymphocytes (e.g., melanoma-melanocyte differentiation antigens such as MART-1, gp 100, tyrosinase, tyrosinase related protein-1, tyrosinase related protein-2, melanocyte-stimulating hormnone receptor; mutated antigens such as beta-catenin, MUM-1, CDK-4, caspase-8, KIA 0205, HLA-A2-R1701; cancer-testes antigens such as MAGE-1, MAGE-2, MAGE-3, MAGE-12, BAGE, GAGE and NY-ESO-1; and non-mutated shared antigens over expressed on cancer such as alpha-fetoprotein, telomerase catalytic protein, G-250, MUC-1, carcinoembryonic antigen, p53, Her-2-neu) as well as antigens recognized by CD4+ lymphocytes (e.g., gp100, MAGE-1, MAGE-3, tyrosinase, NY-ESO-1, triosephosphate isomerase, CDC-27, and LDLR-FUT). See, also, WO 91/02062, U.S. Pat. No. 6,015,567, WO 01/08636, WO 96/30514, U.S. Pat. No. 5,846,538 and U.S. Pat. No. 5,869,445. See also, the list of cancer antigens set forth in Davis et al., “Rational Approaches to Human Cancer immunotherapy”, Journal of Leukocyte Biology 73:3-29 (2003).

In one embodiment, the gene of interest preferably encodes a polypeptide not naturally present in the host cell. In an alternative embodiment, the gene of interest encodes a polypeptide of the host cell in order to, for example, increase the immune response to an encoded immunogen.

Administration

Vaccines according to the invention may either be prophylactic (i.e. to prevent infection) or therapeutic (i.e. to treat infection), but will typically be prophylactic.

The invention also provides a composition of the invention for use as a medicament. The medicament is preferably able to raise an immune response in a mammal (i.e. it is an immunogenic composition) and is more preferably a vaccine.

The invention also provides the use of the compositions of the invention in the manufacture of a medicament for raising an immune response in a mammal. The medicament is preferably a vaccine.

The compositions of the invention may be administered in two or more stages, including priming and boosting. The priming stage of the administration may be designed to expose the subject to the immunogen with the purpose of eliciting a humoral immune response defined by the production of antibody and a cellular immune response that results in the creation of memory T cells. Following priming, the immune system of the subject may generate a response of greater magnitude upon exposure to the same or similar immunogen (the boost dose). The priming stage may include an inactivated host cell comprising a polynucleotide encoding the immunogen. Alternatively, the priming stage may comprise administration of an immunogenic polypeptide or of an alternative polynucleotide delivery method. Such alternative strategies may include a live attenuated virus encoding the immunogen, a live-attenuated bacterium encoding the immunogen, or a polynucleotide encoding the immunogen isolated from any host cell carrier. This priming stage may then be followed by administration of the immunogen using a method different from the priming stage. Preferably, the priming stage comprises administration of the immunogenic polypeptide and the boosting stage comprises administration of the inactivated host cell comprising the polynucleotide encoding the immunogen. Alternatively, the priming stage comprises administration of a host cell comprising a polynucleotide encoding the immunogen and the boosting dose comprises administration of the immunogenic polypeptide.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

The compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral (e.g. tablet, spray), vaginal, topical, transdermnal (e.g. see WO99/27961 or transcutaneous {e.g. see WO02/074244 & WO02/064162}, intranasal (e.g. see WO03/028760), ocular, aural, pulmonary or other mucosal administration. The invention may be used to elicit systemic and/or mucosal immunity.

Dosage treatment can be a single dose schedule or a multiple dose schedule. Multiple doses may be used in a primary immunization schedule and/or in a booster immunization schedule. In a multiple dose schedule the various doses may be given by the same or different routes e.g. a parenteral prime and mucosal boost, a mucosal prime and parenteral boost, etc.

The compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

The vaccine compositions of the invention will comprise a sufficient amount of inactivated host cell so as to generate an immunologically effective immune response to the expressed immunogen. This sufficient amount of inactivated host cell is such that administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the choice of immunogen, the treating doctor's assessment of the medical situation, and other relevant factors. It is expected that the amount will fall in a relatively broad range that can be determined through routine trials.

Further Components of the Composition

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in reference 2.

The vaccines of the invention may be administered in conjunction with other immunoregulatory agents. In particular, compositions of the invention may optionally include include an adjuvant. Preferred further adjuvants include, but are not limited to, one or more of the following set forth below:

A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphoshpates, orthophosphates), sulphates, etc. {e.g. see chapters 8 & 9 of ref. 3}), or mixtures of different mineral compounds, with the compounds taking any suitable form (e.g. gel, crystalline, amorphous, etc.), and with adsorption being preferred. The mineral containing compositions may also be formulated as a particle of metal salt. See ref. 4.

B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See ref. 5.

Complete Freund's adjuvant (CFA) and incomplete Freund's adjuvant (IFA) may also be used as adjuvants in the invention.

C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO 96/33739).

Combinations of saponins and cholesterols can be used to form unique particles called immunostimulating Complexs (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP 0 109 942, WO 96/11711 and WO 96/33739. Optionally, the ISCOMS may be devoid of additional detergent. See ref. 6.

A review of the development of saponin based adjuvants can be found at ref. 7.

C. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, QB-phage (such as coat proteins), GA-phage, fr-phage, AP205 phage, and Ty (such as retrotransposon Ty protein p1). VLPs are discussed further in WO 03/024480, WO 03/024481, and Refs. 8, 9, 10 and 11. Virosomes are discussed further in, for example, Ref. 12

D. Bacterial or Microbial Derivatives

Adjuvants suitable for use in the invention include bacterial or microbial derivatives such as:

(1) Non-toxic Derivatives of Enterobacterial lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Ref. 13.

(2) Lipid A Derivatives

Lipid A derivatives include derivatives of lipid A from Escherichia coli such as OM-174. OM-174 is described for example in Ref. 14 and 15.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See ref. 16, WO 02/26757 and WO 99/62923 for examples of possible analog substitutions. The adjuvant effect of CpG oligonucleotides is further discussed in Refs. 17, 18, WO 98/40100, U.S. Pat. No. 6,207,646, U.S. Pat. No. 6,239,116, and U.S. Pat. No. 6,429,199.

The CpG sequence may be directed to TLR9, such as the motif GTCGTT or TTCGTT. See ref. 19. The CpG sequence may be specific for inducing a Th1 immune response, such as a CpG-A ODN, or it may be more specific for inducing a B cell response, such a CpG-B ODN. CpG-A and CpG-B ODNs are discussed in refs. 20, 21 and WO 01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 22, 23, 24 and WO 03/035836.

(4) ADP-ribosylating Toxins and Detoxified Derivatives Thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO 95/17211 and as parenteral adjuvants in WO 98/42375. Preferably, the adjuvant is a detoxified LT mutant such as LT-K63.

E. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

F. Bioadhesives and Mucoadhesives

Bioadhesives and mucoadhesives may also be used as adjuvants in the invention. Suitable bioadhesives include esterified hyaluronic acid microspheres (Ref. 25) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g., ref. 26.

G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

H. Liposomes

Examples of liposome formulations suitable for use as adjuvants are described in U.S. Pat. No. 6,090,406, U.S. Pat. No. 5,916,588, and EP 0 626 169.

I. Polyoxyethylene Ether and Polyoxyethylene Ester Formulations

Adjuvants suitable for use in the invention include polyoxyethylene ethers and polyoxyethylene esters. Ref. 27. Such formulations further include polyoxyethylene sorbitan ester surfactants in combination with an octoxynol (Ref. 28) as well as polyoxyethylene alkyl ethers or ester surfactants in combination with at least one additional non-ionic surfactant such as an octoxynol (Ref. 29).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosphazene (PCPP)

PCPP formulations are described, for example, in Ref. 30 and 31.

K. Muramyl Peptides

Examples of muramyl peptides suitable for use as adjuvants in the invention include N-acetyl-muramyl-L-threonyl-D-isoglutamine (thr-MDP), N-acetyl-norrnuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

L. lmidazoguinolone Compounds.

Examples of imidazoquinolone compounds suitable for use adjuvants in the invention include Imiquamod and its homologues, described further in Ref. 32 and 33.

The invention may also comprise combinations of aspects of one or more of the adjuvants identified above. For example, the following adjuvant compositions may be used in the invention:

(1) a saponin and an oil-in-water emulsion (ref. 34);

(2) a saponin (e.g., QS21)+a non-toxic LPS derivative (e.g., 3dMPL) (see WO 94/00153);

(3) a saponin (e.g., QS21)+a non-toxic LPS derivative (e.g., 3dMPL)+a cholesterol;

(4) a saponin (e.g. QS21)+3dMPL+IL-12 (optionally+a sterol) (Ref. 35); combinations of 3dMPL with, for example, QS21 and/or oil-in-water emulsions (Ref. 36);

(5) SAF, containing 10% Squalane, 0.4% Tween 80, 5% pluronic-block polymer L121, and thr-MDP, either microfluidized into a submicron emulsion or vortexed to generate a larger particle size emulsion.

(6) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and

(7) One or More Mineral Salts (Such as an Aluminum Salt)+a Non-toxic Derivative of LPS (Such as 3dPML).

Aluminum salts and MF59 are preferred adjuvants for parenteral immunization. Mutant bacterial toxins are preferred mucosal adjuvants.

The above-described polypeptide based adjuvants may be administered directly as peptides, or, alternatively, may be administered along with the immunogen as a polynucleotide encoding the adjuvant associated with the inactivated host cell.

Immunostimulatory oligonucleotides may also be incorporated into inactivated host cells for delivery with the compositions of the invention or administered unassociated with the inactived host cells.

The composition may further include an antibiotic.

### EXAMPLE

Preparation of Inactivated Host Cells

Escherichia coli DH5α (Invitrogen, Calif.) and Shigella flexneri 1207 (obtained from University of Maryland, Kotloff et al. 2000) hosts were grown in LB medium. The cells were transformed with either pCM.gag (for E. coli) or pCMVH.gag (for Shigella) DNA vaccine plasmid (Xu et al, 2003). The two polynucleotide vectors carry the same HIV-1 SF2 Gag gene controlled by pCMV promoter, a mammalian expression element; and different antibiotic selection markers: kanamycin resistance gene (for pCMV.gag) and ampicilin resistance gene (for pCMVH.gag) (Xu et al, 2003). After the host cells cells were grown to late log phase and then washed 3 times with saline solution (0.9% NaCl), they were inactivated with treatment of heat at 50° C. for 30 minutes and diluted as required for use in animal.

Immunization with Inactivated Host Cells

CB6F1 mice were purchased from Charles River Breeding Laboratories (Wilmington, Mass.) and housed at an AALAC-accredited facility (Chrion, Emeryville, Calif.). Mice were lightly sedated and injected in 2×50 μl volume of inactivated host cells (made as described above) into the tibialis anterior muscle of each mouse leg. Three weeks after immunization, mice were injected with 1×10⁷ PFU of a recombinant vaccinia virus (rvv) encoding the HIV-1 SF2 gag-pol genes (rvvgag-poo) (zur Megede et al., 2000). Five days after rvv challenge, mouse spleens were collected and processed to a single-cells suspension. Gag-specific immune responses were measured by interferon-γ (IFN-γ) production after stimulation with an H-2^{d}-restricted gag derived peptide (sequence: AMQMLKETI; positioning at the region of 199-207 amino acid residues in HIV Gag p24 protein), according to zur Megede et al (2000). This peptide sequence was not found in the bacterial genomes with computer search using Blast program.

TABLE 1
Cellular response after immunization of killed recombinant
bacterial cells carrying HIV gag DNA vaccines in mice

| | INF-γ production |
|---|---|
| Vaccine | (pg/10⁶ spleen cells) |
| Killed E. coli (pCMV.gag) (10⁻³OD₆₀₀) | 96.4 |
| Killed Shigella (pCMVH.gag) (10⁻³OD₆₀₀) | 313 |
| Killed Shigella (pCMVH.gag) (10⁻⁴OD₆₀₀) | 175 |
| Saline | 0 |

As shown in Table 1, after immunization with the inactivated host cells carrying polynucleotides encoding HIV gag proteins, the specific cell mediated response against HIV derived peptide p7g, showing the production of INF-γ by the immunized mouse spleen cells upon stimulation with the peptide, was detected.

### CITED REFERENCES:

1. Sutherland, “Biosynthesis and composition of gram-negative bacterial extracellular and wall polysaccharides”, Ann. Rev. Microbiol. (1985) 39: 243-270.
2. Gennaro (2000) Remington: The Science and Practice of pharmacy. 20th ed., ISBN: 0683306472.
3. Vaccine design:the subunit and adjuvant approach (1995) Powell & Newman. ISBN 0-306-44867-X.
4. WO00/23105.
5. WO90/14837.
6. WO00/07621.
7. Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.
8. Niikura et al., “Chimeric Recombinant Hepatitis E Virus-Like Particles as an Oral Vaccine Vehicle Presenting Foreign Epitopes”, Virology (2002) 293:273-280.
9. Lenz et al., “Papillomarivurs-Like Particles Induce Acute Activation of Dendritic Cells”, Journal of Immunology (2001) 5246-5355.
10. Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338.
11. Gerber et al., “Human Papillomavrisu Virus-Like Particles Are Efficient Oral Immunogens when Coadministered with Escherichia coli Heat-Labile Entertoxin Mutant R192G or CpG”, Journal of Virology (2001) 75(10):4752-4760.
12. Gluck et al., “New Technology Platforms in the Development of Vaccines for the Future”, Vaccine (2002) 20:B10-B16.
13. Johnson et al. (1999) Bioorg Med Chem Lett 9:2273-2278.
14. Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491.
15. Pajak, et al., “The Adjuvant OM-174 induces both the migration and maturation of murine dendritic cells in vivo”, Vaccine (2003) 21:836-842.
16. Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400.
17. Krieg, “CpG motifs: the active ingredient in bacterial extracts?”, Nature Medicine (2003) 9(7): 831-835.
18. McCluskie, et al., “Parenteral and mucosal prime-boost immunization strategies in mice with hepatitis B surface antigen and CpG DNA”, FEMS Immunology and Medical Microbiology (2002) 32:179-185.
19. Kandimalla, et al., “Toll-like receptor 9: modulation of recognition and cytokine induction by novel synthetic CpG DNAs”, Biochemical Society Transactions (2003) 31 (part 3): 654-658.
20. Blackwell, et al., “CpG-A-Induced Monocyte IFN-gamma-Inducible Protein-10 Production is Regulated by Plasmacytoid Dendritic Cell Derived FN-alpha”, J. Immunol. (2003) 170(8):4061-4068.
21. Krieg, “From A to Z on CpG”, TRENDS in Immunology (2002) 23(2): 64-65.
22. Kandimalla, et al., “Secondary structures in CpG oligonucleotides affect immunostimulatory activity”, BBRC (2003) 306:948-953.
23. Kandimalla, et al., “Toll-like receptor 9: modulation of recognition and cytokine induction by novel synthetic GpG DNAs”, Biochemical Society Transactions (2003) 31(part 3):664-658.
24. Bhagat et al., “CpG penta-and hexadeoxyribonucleotides as potent immunomodulatory agents” BBRC (2003) 300:853-861.
25. Singh et al. (2001) J. Cont. Rele. 70:267-276.
26. WO99/27960.
27. WO99/52549.
28. WO 01/21207.
29. WO01/21152.
30. Andrianov et al., “Preparation of hydrogel microspheres by coacervation of aqueous polyphophazene solutions”, Biomaterials (1998) 19(1-3):109-115.
31. Payne et al., “Protein Release from Polyphosphazene Matrices”, Adv. Drug. Delivery Review (1998) 31(3):185-196.
32. Stanley, “Imiquamod and the imidazoquinolone: mechanism of action and therapeutic potential” Clin Exp Dermatol (2002) 27(7):571-577.
33. Jones, “Resiquimod 3M”, Curr Opin Investig Drugs (2003) 4(2):214-218.
34. WO99/11241.
35. WO98/57659.
36. European patent applications 0835318, 0735898 and 0761231.

1. A method for in vivo expression of an immunogen comprising:
administering an inactivated host cell to a mammal, wherein said host cell comprises a polynucleotide encoding an immunogen, and wherein the immunogen is expressed in vivo by the mammalian cells.

2. The method of claim 1 wherein the host cell is inactivated by heat treatment.

3. The method of claim 1 wherein the host cell is inactivated by ultra-violet light exposure.

4. The method of claim 1 wherein the host cell is inactivated by hydrogen peroxide treatment.

5. The method of claim 1 wherein a plasmid comprises the polynucleotide encoding the immunogen.

6. The method of claim 1 wherein the polynucleotide encoding the immunogen is incorporated into the host cell genome.

7. The method of claim 1 wherein the expressed immunogen generates an immune response in the mammal.

8. A method of generating an immune response in a mammal comprising:
administering an inactivated host cell to said mammal, wherein said inactivated host cell comprises a polynucleotide encoding an immunogen.

9. The method of claim 8 wherein the host cell is inactivated by heat treatment.

10. The method of claim 8 wherein the host cell is inactivated by ultraviolet light exposure.

11. The method of claim 8 wherein the host cell is inactivated by hydrogen peroxide treatment.

12. The method of claim 8 wherein a plasmid comprises the polynucleotide encoding the immunogen.

13. The method of claim 8 wherein the polynucleotide encoding the immunogen is incorporated into the host cell genome.

14. A composition comprising an inactivated host cell that comprises a polynucleotide encoding an immunogen.

15. The composition of claim 14 further comprising a pharmaceutically acceptable solution.

16. The composition of claim 14 further comprising a pharmaceutically acceptable carrier.

17. A method of making an inactivated host cell comprising:
(i) transforming a host cell with a vector comprising a polynucleotide encoding an immunogen; and
(ii) inactivating the host cell.

18. The method of claim 17 wherein the step of inactivating is performed by heat treatment.

19. The method of claim 17 wherein the step of inactivating is performed by ultraviolet light exposure.

20. The method of claim 17 wherein the step of inactivating is performed by hydrogen peroxide treatment.

21. The method of claim 17 wherein the inactivated host cells are detoxified.

22. The method of claim 21 where the detoxification is by genetic manipulation of the host cells′ lipopolysaccharide synthesis genes.
